(19) United States
(12) Patent Application Publication
(10) Pub. No.: US 2026/0224369 A1
(43) Pub. Date: Aug. 6, 2026

(54) Orthopedic Dowel Fusion Devices, Systems, and Methods

(71) Applicant: Innovative Midfoot Solutions, Inc., Aurora, CO (US)

(72) Inventors: Gregory C Berlet, Worthington, OH (US); Stephen A. Brigido, Bethlehem, PA (US); Murray J. Penner, Vancouver (CA); Christopher E. Banas, Breckenridge, CO (US); Jeremy H. Morgan, Missoula, MT (US); Jeffrey N. Steinmetz, Arvada, CO (US)

(73) Assignee: Innovative Midfoot Solutions, Inc., Aurora, CO (US)

(21) Appl. No.: 19/431,438

(22) Filed: Dec. 23, 2025

Related U.S. Application Data
(63) Continuation-in-part of application No. 18/459,029, filed on Aug. 30, 2023.
(60) Provisional application No. 63/738,735, filed on Dec. 24, 2024.
(60) Provisional application No. 63/738,736, filed on Dec. 24, 2024.
(60) Provisional application No. 63/738,738, filed on Dec. 24, 2024.
(60) Provisional application No. 63/738,739, filed on Dec. 24, 2024.
(60) Provisional application No. 63/738,741, filed on Dec. 24, 2024.
(60) Provisional application No. 63/738,912, filed on Dec. 26, 2024.
(60) Provisional application No. 63/738,927, filed on Dec. 26, 2024.
(60) Provisional application No. 63/738,929, filed on Dec. 26, 2024.
(60) Provisional application No. 63/738,920, filed on Dec. 26, 2024.

(51) Int. Cl. A61F 2/42; A61F 2/30; A61F 2/46

(52) U.S. Cl. CPC A61F 2/4225; A61F 2/30749; A61F 2/4606; A61F 2002/30011; A61F 2002/3021; A61F 2002/30622

(57) ABSTRACT

Devices, systems and methods for joint fusion or arthrodesis of articular joints in treatment orthopedic disorders, including a dowel fusion kit, a frustoconical dowel configured to be implanted within the articular space of an articular joint and secured within the joint by either or both of a compression screw and/or a staple.

### CROSS-REFERENCE TO RELATED APPLICATIONS

This application is continuation-in-part of U.S. patent application Ser. No. 18/459,029 filed Aug. 30, 2023 and claims priority to U.S. Provisional Applications Nos. 63/738,735. 63/738,736, 63/738,738, 63/738,739, and 63/738,741, all filed Dec. 24, 2024 and U.S. Provisional Applications Nos. 63/738,912, 63/738,920, 63/738,927, and 63/738,929, all filed Dec. 26, 2024.

### BACKGROUND OF THE INVENTION

The present disclosure pertains generally to instrumentation, devices, and methods for surgical treatment of orthopedic disorders such as osteoarthritis, spacing disorders, or alignment disorders. More specifically, the present disclosure pertains to dowel implants for orthopedic arthrodesis procedures in anatomical joints. The devices, instrumentation, and methods of the present disclosure also have relevance and pertains to all transverse or vertically oriented joint fusions, including, without limitation in the hand, wrist, foot, and/or ankle.

### SUMMARY OF THE INVENTION

It is an object of the present disclosure to provide instrumentation, devices, and methods for surgical treatment of orthopedic joint disorders.

It is another object of the present disclosure to provide instrumentation, devices, and methods for joint fusion in the foot and hand anatomy.

It is a further object of the present disclosure to instrumentation, devices, and methods for mid-foot joint arthrodesis.

It is still another object of the present disclosure to provide instrumentation, devices, and methods for fusion or arthrodesis of the tarsometatarsal joints, navicular cuneiform joints, the metartasocuneiform joints, the talon-navicular joints, the intercuneiform joints, the subtalar joint, the calcaneocuboid joint, and/or the metatarsophalangeal joints of the foot.

### BRIEF DESCRIPTION OF THE DRAWINGS

FIG. 1 is a perspective view of a first variant of an arthrodesis dowel in accordance with the present disclosure.

FIG. 2 is a perspective view of a second variant of an arthrodesis dowel in accordance with the present disclosure.

FIG. 3 is a perspective view of a third variant of an arthrodesis dowel in accordance with the present disclosure and illustrating dimensional variables common to all arthrodesis dowel variants of the present disclosure.

FIGS. 4A and 4B are perspective views of the third variant illustrating different dimensional configurations of the third variant of the present disclosure.

FIGS. 4C and 4D are perspective views of a fourth variant of the arthrodesis dowel of the present disclosure illustrating different dimensional configurations thereof.

FIGS. 4E and 4F are perspective views of a fifth variant of the arthrodesis dowel of the present disclosure illustrating different dimensional configurations thereof.

FIG. 4G is a perspective view of a sixth variant of the arthrodesis dowel of the present disclosure illustrating a multi-faceted taper configuration thereof.

FIG. 4H is a perspective view of a seventh variant of the arthrodesis dowel of the present disclosure illustrating a stepped taper configuration thereof.

FIG. 4I is a perspective view of an eighth variant of the arthrodesis dowel according to the present disclosure.

FIG. 4J is a perspective view of a ninth variant of the arthrodesis dowel according to the present disclosure.

FIG. 4K is a perspective view of a tenth variant of the arthrodesis dowel according to the present disclosure.

FIG. 4M is a perspective view of an eleventh variant of the arthrodesis dowel according to the present disclosure.

FIG. 4N is a perspective view of a twelfth variant of the arthrodesis dowel according to the present disclosure.

FIG. 4O is a perspective view of a thirteenth variant of the arthrodesis dowel according to the present disclosure.

FIG. 5A is a cross-sectional view taken along line 5A-5A of FIG. 4F.

FIG. 5B is a perspective view of fourteenth variant of the arthrodesis dowel according to the present disclosure.

FIG. 5C is a side elevational view of the fourteenth variant of the arthrodesis dowel according to the present disclosure.

FIG. 5D is an axially transverse cross-sectional view of the arthrodesis dowel according to the present disclosure illustrating porous and non-porous morphology of the material of the any of the variants of the arthrodesis dowel of the present disclosure.

FIG. 6 is a perspective view of a joint finder device in accordance with the system of the present disclosure.

FIG. 7 is a perspective view of a guide instrument in accordance with the system of the present disclosure.

FIG. 8 is a perspective view of a K-wire in accordance with the system of the present disclosure.

FIG. 9 is a perspective view of a drilling tool in accordance with the present disclosure.

FIG. 10 is a perspective view of a dowel delivery tool in accordance with the present disclosure.

FIGS. 11A to 11K are sequential views of a first method of dowel joint arthrodesis in accordance with the present disclosure.

FIGS. 12A to 12J are sequential views of a second method of dowel joint arthrodesis in accordance with the present disclosure.

FIG. 13 is a perspective view illustrating use of the guide instrument, the K-wires, and drilling tool in accordance with the present disclosure.

FIG. 14 is a perspective view illustrating implanting a dowel in a joint articular space employing the guide instrument, K-wires, and dowel delivery tool in accordance with the present disclosure.

FIG. 15 is a perspective view illustrating an arthrodesis fixation guide in operable association with the guide instrument, K-wires, dowel delivery tool, and dowel in accordance with the present disclosure.

FIG. 16 is a perspective view illustrating the arthrodesis fixation guide coupled to an introducer guidewire.

FIG. 17 is a perspective view illustrating a canulated drill bit passing over the introducer guidewire creating a drilled bore passing through proximal and distal bones of a joint and a fixation opening in a dowel in accordance with the present disclosure.

FIG. 18 is a perspective view depicting a cannulated compression screw passing over the introducer guidewire and into the drilled bore through the dowel and the proximal and distal bones of the joint.

FIG. 19 is a perspective view depicting a fully seated compression screw across the joint.

FIG. 20 is a perspective view illustrating use of the joint finder device and guide instrument in accordance with the present disclosure.

FIG. 21 is a top elevational view illustrating use of the guide instrument in accordance with the present disclosure.

FIG. 22 is a perspective view of a first variant of the guide instrument illustrated with a drilling tool in accordance with the present disclosure.

FIG. 23 is a perspective view of the first variant of the guide instrument with an associated dowel and dowel delivery tool and a fixation guide mating collar in accordance with the present disclosure.

FIG. 24 is a perspective view of a first variant of the arthrodesis fixation guide illustrating its engagement with the mating collar, a dowel delivery tool, and a drill bit.

FIG. 25 is a perspective view of the first variant of the arthrodesis fixation guide illustrating its engagement with the mating collar, a dowel delivery tool, a drill bit, and a guidewire.

FIGS. 26A to 24D sequentially illustrate placement of a fixation screw over a guidewire and through the fixation opening in the dowel and across a joint.

FIG. 27 is a perspective view of a fixation staple across a joint securing a dowel in articular space of the joint.

FIG. 28 is a perspective view of a fixation staple placed across a joint securing a dowel and fixation screw in the articular space of the joint in accordance with the present disclosure.

FIG. 29A is a perspective view of an arthrodesis dowel of the present disclosure implanted into a mid-foot joint space.

FIG. 29B is a perspective view of an arthrodesis dowel secured by a fixation staple across a mid-foot joint space in accordance with the present disclosure.

FIGS. 30A to 30C are side elevational views of alternate size joint sizing gauges in accordance with the present disclosure.

FIG. 31 is an end elevational view of a joint sizing gauge in accordance with the present disclosure.

FIG. 32 is a fragmentary side elevational view of a distal portion of a joint finder instrument in accordance with the present disclosure.

FIG. 33A is a side elevational view of a variant of the joint finder in accordance with the present disclosure.

FIG. 33B is a second side elevational view of the variant of the joint finder of FIG. 33A.

FIG. 33C is a third side elevational view of a variant of the joint finder of FIG. 33A.

FIG. 33D is a fourth side elevational view of a variant of the join finder of FIG. 33A.

FIG. 34A is a side elevational view of a surgical guide instrument in accordance with the present disclosure.

FIG. 34B is a top elevational view of the surgical guide instrument illustrating K-wire apertures in a first spaced position in accordance with the present disclosure.

FIG. 34C is a top elevational view of the surgical guide instrument illustrating K-wire apertures in a second spaced position in accordance with the present disclosure.

FIG. 35 is a side elevational view of a drill bit in accordance with the present disclosure.

FIG. 36A is a side elevational view of a drill bit instrument in accordance with the present disclosure.

FIG. 36B is a fragmentary side elevational view taken along circle 36B of FIG. 36A illustrating a spacing collar in a first position in accordance with the present disclosure.

FIG. 36C is a fragmentary side elevational view of the drill bit instrument of the present disclosure illustrating the spacing collar in a second position.

FIG. 37A is a side elevational view of a dowel driver instrument in accordance with the present disclosure.

FIG. 37B is a longitudinal cross-sectional view taken along line 37B-37B of FIG. 37A.

FIG. 38A is a perspective view of the dowel driver instrument in a first delivery state.

FIG. 38B is a perspective view of the dowel driver instrument in a second delivery state.

FIG. 38C is a perspective view of the dowel driver instrument in a third delivery state.

FIGS. 39A-39C are sequential views of a dowel arthrodesis placement method in a mid-foot joint in accordance with the present disclosure.

### DETAILED DESCRIPTION OF THE PREFERRED EMBODIMENTS

For purposes of clarity, the following terms used in this patent application will have the following meanings:
The terminology used herein is for the purpose of describing example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the order discussed or illustrated, unless specifically identified as an order of performance. It is also to be understood that additional or alternative steps may be employed.
When an element or layer is referred to as being “on,” “engaged,” “connected,” or “coupled” to or with another element, it may be directly on, engaged, connected, or coupled to the other element or layer, or intervening elements or layers may be present. In contrast, when an element is referred to as being “directly on,” “directly engaged to,” “directly connected to,” or “directly coupled to” or with another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

“Substantially” is intended to mean a quantity, property, or value that is present to a great or significant extent and less than, more than or equal to total. For example, “substantially vertical” may be less than, greater than, or equal to completely vertical.

“About” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

Recitation of ranges of values herein are merely intended to serve as a shorthand method of referring individually to each separate value falling within the recited range, unless otherwise indicated herein, and each separate value is incorporated into the specification as if it were individually recited herein.

References to “embodiment” or “variant”, e.g., “one embodiment,” “an embodiment,” “example embodiment,” “various embodiments,” etc., may indicate that the embodiment(s) or variant(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment or variant, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The term “frustoconical” is intended to mean a truncated cone shape having a base, a truncation, and side walls that either 1) lie in a plane rotated about the truncation of the cone, 2) have plural angled side walls along the longitudinal axis of the truncated cone that each circumscribe a different angular orientation, or 3) form plural stepped side walls of decreasing or increasing diameter from the truncation of the cone to the base of the cone.

The term “material” is intended to refer to encompass biocompatible materials, including metals, ceramics, polymers, composites, and combinations or hybrids thereof.

As used in this application the term “layer” is intended to mean a substantially uniform material limited by interfaces between it and adjacent other layers, substrate, or environment.

The terms “circumferential” or “circumferential axis” is intended to refer to the radial direction of a tubular, cylindrical or annular material or to the Y-axis of a polygonal material.

The terms “longitudinal,” “longitudinal axis,” or “tube axis” are intended to refer to an elongate aspect or axis of a material or to the X-axis of the material.

The term “medial” is intended to denote a position towards the midline of the body.

The term “lateral” is intended to mean a position away from the midline of the body.

The term “plantar” is intended to refer to a position toward the sole of the foot.

The term “dorsal” is intended to refer to a position away from the sole of the foot.

The term “mid-foot” is intended to mean the medial column section of the human foot between the hindfoot and forefoot and includes five of the seven tarsal bones, i.e., navicular, cuboid, and three cuneiform bones.

The term “hindfoot” is intended to mean the posterior section of the human foot comprising the region of the talus and calcaneus bones.

The term “forefoot” is intended to mean the anterior section of the human foot comprising the metatarsal and phalangeal bones.

The terms “tarsometatarsal” or “TMT” are intended to relate to the articulations between the tarsal and metatarsal bones of the foot and the ligaments in relation thereto.

The terms “metatarsocuneiform” or “MC” are intended to relate to the joint or articulations between the metatarsal and cuneiform bones of the human foot and the ligaments in relation thereto.

The terms “navicular cuneiform” or “NC” are intended to refer to the joint or articulations in the human mid-foot consisting of the tarsal, navicular, and the medial, middle, and lateral cuneiform bones.

The terms “talon-navicular” or “TN” are intended to refer to the joint or articulations in the human mid-foot consisting of the talus and navicular bones.

The terms “subtalar joint” or “STJ” is intended to refer to the joint or articulations in the human foot consisting of the talus bone and the calcaneus bone, as well as the interosseous talocalcaneal ligament.

The terms “calcaneocuboid” or “CC” are intended to refer to the joint or articulations in the human foot between the calcaneus and the cuboid bone.

The terms “metatarsophalangeal” or “MTP” are intended to refer to the joint or articulations of the human foot between the metatarsal and phalangeal bones.

The term “interphalangeal” is intended to refer to the joint or articulations of the human foot between phalangeal bones.

The term “arthrodesis” is intended to refer to a surgical immobilization of a joint by fusion of the adjacent bones. The terms “arthrodesis” and “fusion” are used synonymously in the present application.

The various embodiments of the arthrodesis dowel and system for joint arthrodesis of the present disclosure will be described in greater detail with reference to the accompanying drawings. It is not intended, nor should it be construed, that the scope of the embodiments be limited to the described features, materials, physical or dimensional specifications, arrangements, or uses. Rather, it is intended that the scope of the embodiments described be confined only to the claims appended hereto or as may be amended during prosecution of this application.

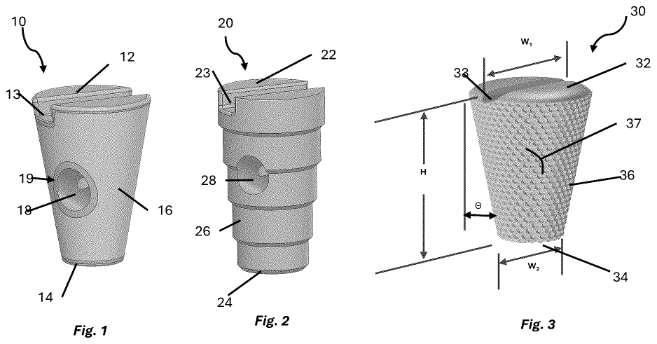

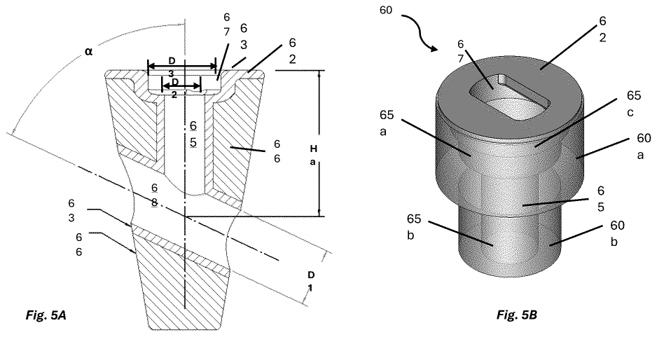

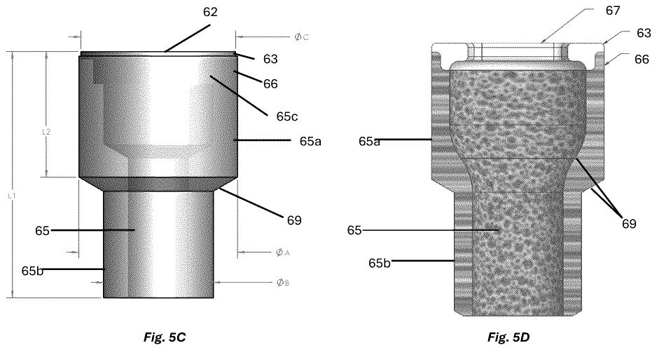

With respect to the arthrodesis dowel of the present disclosure, variants of the arthrodesis dowel are illustrated in FIGS. 1-3, 4A-4O, and 5A-5D. FIG. 1 depicts a first dowel variant 10 having a substantially frustoconical shape defined by a proximal or upper surface 12 and a distal or lower surface 14 and distally tapering side walls 16 between the proximal surface 12 and the distal surface 14. Side walls 16 may be distally tapering, proximally tapering, or be non-cylindrical. For purposes of the present application, for the mid-foot anatomy where surgical access is achieved through a dorsal approach, the side walls 16 are distally tapering side walls 16, and are referred to as such herein, without limiting the alternative configurations of the side walls 16. The proximal or upper surface 12 has a depression 13 that extends substantially diametrically across the upper surface 12 is open at diametrically opposite sides of the side wall 16. An optional angled bore 18 is provided that extends diametrically across the first dowel variant 10 at an intermediate point along a length of the distally tapering side walls 16. The proximal surface 12, the distal surface 12, the depression 13, and the optional angled bore 18 are optionally formed of a non-porous material 63, while the remainder of the dowel 10 is formed of a porous material 66, as exemplified in FIGS. 5A and 5D.

FIG. 2 depicts a second dowel variant 20, which is also formed as a generally frustoconical shape having an upper or proximal surface 22, a lower or distal surface 24, a depression 22 formed in the upper or proximal surface 22 that extends diametrically across the upper surface, side walls 26, and a transverse angled bore 28 passing diametrically through the side walls 26 intermediate the upper surface 22 and the lower surface 24. In the second dowel variant 20, the side walls 26 taper distally in a series of circumferential steps having decreasing diameters toward the distal surface 24. This stepped configuration of the side walls 26 provides greater surface area on the side walls 26 for osteo-integration or bone ingrowth to fix the second dowel variant 20 in the fused joint. The proximal surface 22, the distal surface 22, the depression 23, and the angled bore 28 are optionally formed of a non-porous material, while the remainder of the dowel 20 is formed of a porous material.

FIG. 3 illustrates a third dowel variant 30 which also has a proximal or upper surface 32, a lower or distal surface 34, distally tapering side walls 36, and an optional diametrically extending depression 33 across the upper surface 32. The side walls 36 have a textured surface 37 configured to promote osteo-integration to fixate the third dowel variant 30 in the fused joint. Unlike first dowel variant 10 and second dowel variant 20, third dowel variant 30 may or may not have a transverse angled bore passing through the dowel 30 intermediate the proximal surface 32 and the distal surface 34.

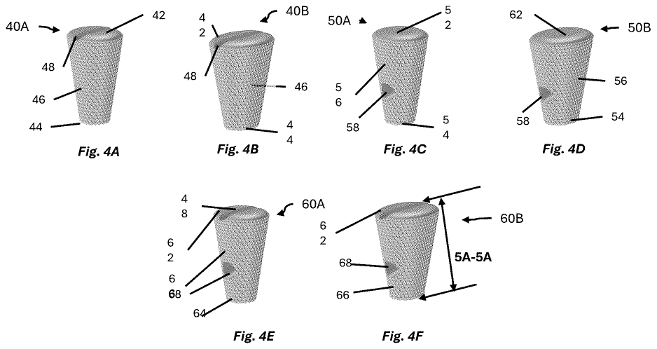

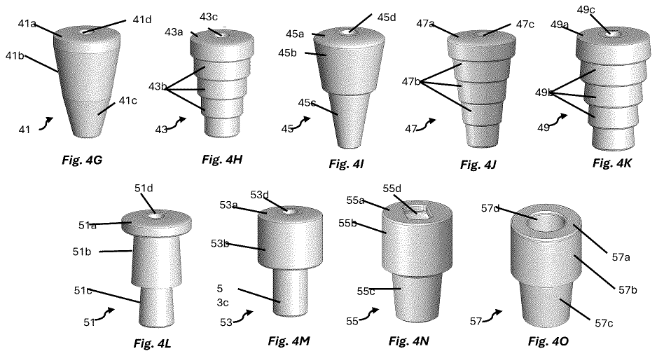

FIGS. 4A to 5D depict further dowel variants 40A, 40B, 50A, 50B, 60A, 60B, 41, 43, 45, 47, 49, 51, 53, 55, and 57 (unless otherwise specified, all dowel variants will be referred to as “dowel 10”) in which each has an upper or proximal transverse surface (“proximal surface), e.g., 41a, 43a, 45a, 47a, 49a, 51a, 53a, 55a, 57d, and a receiver opening, e.g., 41d, 43c, 45d, 47c, 49c, 51d, 53d, 55d, 57d, and /r a recess, e.g., 42, 52, 48, distally tapering side walls e.g., 41b, 41c, 43b, 45b, 45c, 47b, 49b, 51b, 51c, 53b, 53c, 55b, 55c, 57b, 57c, and optional transverse angled bores passing diametrically through the dowel variants intermediate the proximal surfaces and the distal surfaces. The distally tapering side walls may defined alternatively by i) a normal plane rotated about the circumference between the proximal surface and the distal surface; ii) a multi-taper geometry as exemplified in FIG. 4G, in which the walls have a proximal section 41b that tapers with a first angular orientation from an upper portion 41a of the dowel and a distal section 41c with a second angular orientation; iii) a stepped geometry in which a proximal section has a first diameter and a distal section has a second diameter; iv) a stepped geometry wherein both the proximal section and the distal section taper have stacked ring structures 49b of reducing diameters toward the distal end of the dowel and each of the stacked ring structures 49b flare diametrically outward along their longitudinal axis as exemplified in FIGS. 4K and 4L; v) a stepped geometry wherein the proximal section 41b, 45b and the distal section 41c, 45c taper diametrically inward along their longitudinal axes, as exemplified in FIGS. 4G and 4I; vi) a stepped geometry the proximal section 53b is not tapered and the distal section is distally tapered 55c, as exemplified in FIG. 4N; or vii) a stepped geometry wherein the proximal section 53a is not tapered and the distal section 53c is not tapered, as exemplified in FIG. 4M. As shown in FIGS. 4H, 4J, and 4K, there may also be one or more intermediate sections between the proximal section and the distal section, wherein each of the one or more intermediate sections have decreasing diameters toward the distal section, and each of the one or more intermediate sections may have no taper, an inward taper, or an outward taper and the longitudinal length of each of the one or more intermediate sections. The upper or proximal transverse surface may be formed as a cap member having the receiver opening formed therein. The cap member may be integrally formed during manufacture of the dowel or may be fixedly coupled to the dowel during manufacture. It is expressly intended that any of the foregoing combinations of dowel wall profiles are included in the scope of variants of the dowel intended by the present disclosure.

As shown in FIGS. 5A to 5D, each of the dowel variants has a bore 65 passing along a central longitudinal axis of the dowel variant into and through the upper or proximal surface 62 and into the dowel 60. The bore 65 may communicate with and terminate at the transverse angled bore 68 or, alternatively, extend along an entire longitudinal axis of the dowel 60 and terminate with an opening at a distal end of dowel 60. As shown in FIG. 5A, bore 65 may be a bore having a constant diameter. Alternatively, as shown in FIGS. 5B and 5C, bore 65 may be configured to have a first larger diameter 65a in a proximal section 60a of the dowel 60 and a second smaller diameter 65b in a distal section 60b of dowel 60. Both the bore 65 and the outer surface of the dowel 60 have a tapered or stepped transition 69 between the proximal section 60a and the distal section 60b. Optionally, the bore 65 may have a third large diameter section 65c at the most proximal aspect of bore 65 that is adjacent to a distal aspect of the proximal surface 62. Bore 65 has a proximal opening that communicates with a coupling aperture 67 in the proximal surface 62 of the dowel 60. The third large diameter section 65c has a greater diameter than the first larger diameter 65a of bore 65 and accommodates engagement of a dowel delivery device 110, 900, with the coupling aperture 67. Coupling aperture 67 is configured to removably couple the dowel 60 to the dowel delivery device 110 or 900, shown in FIGS. 10 and 37A to 38C, and discussed further infra.

Optionally, the outer wall may have a porous surface topography configured to stimulate osteointegration of the dowel into the joint. The surface topography may be imparted by mechanical means, such as electrical discharge machining (EDM) or laser texturing, chemical texturing, magnetron sputtering, physical vapor deposition, photolithography, or three-dimensional (3D) printing. The imparted surface topography may consist of a roughened topography with raised and recessed portions or may be porous; in either case the imparted surface topography may have a regular, irregular, or random pattern to the roughness or porosity. The surface topography may be present on the walls of the dowel 10 or may fully or partially extend into or through the walls of the dowel 10. With any of the foregoing methods of imparting surface topography roughness, a regular or irregular surface topography pattern may be created on the side walls 36. An example of a regular surface pattern is a lattice structure wherein the lattice structure is capable of being altered in both size and geometry on the side walls 36, The surface topography roughness of the side walls 36 may be classified based upon average roughness (Rₐ) into smooth (average roughness Rₐ<0.5 μm), machined/minimal (Rₐ=0.5-1 μm), moderate (Rₐ=1-2 μm) and rough (Rₐ>2 μm). Where 3D printing is employed, the lattice structure may, optionally, be formed in a gyroid or lidinoid configuration.

FIG. 3 also illustrates variable dimensions that are common to all dowel variants of the present disclosure. W₁ defines the width or diameter of the proximal or upper surface, W₂ defines the width or diameter of the lower or distal surface, H defines the height or distance between the proximal or upper surface and the distal or lower surface, and θ defines the angle formed by the distally tapering side walls between the upper or proximal surface and the distal or lower surface. Depending upon the joint anatomy of the joint to be fused and the depth of the bone surfaces of the joint, optionally, in each of the dowel variants, W₁ may be between about 7 mm and about 12 mm, W₂ may be between about 5 mm to about 9 mm, and H may be between about 12 mm and 18 mm.

The non-porous material and/or the porous material of each of the dowel variants of the present disclosure may be selected from the group of biocompatible materials including titanium, stainless steel, cobalt chrome, nickel-titanium alloys, tantalum, polyetheretherketone (PEEK), magnesium, silicon nitride, alumina, zirconia, poly(methyl methacrylate) (PMMA), polylactic acid (PLA), carbon fiber-polyetherketone, or carbon fiber-ultrahigh molecular massed polyethylene.

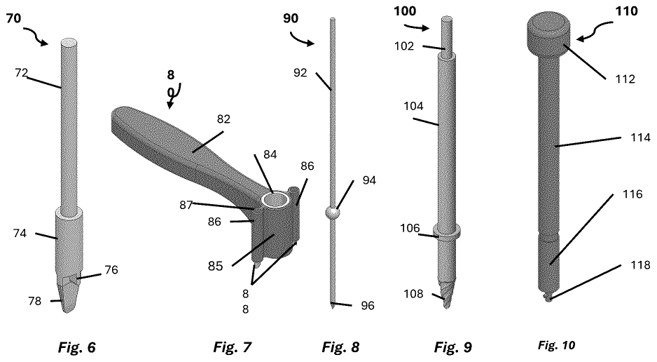

FIGS. 6 to 10 depict different arthrodesis instruments useful in an arthrodesis procedure to deliver each of the dowel variants of the present disclosure.

FIG. 6 illustrates a joint probe 70 that is inserted within the articular space between two opposing bones of the joint. The joint probe 70 includes an elongate shaft 72, a blade 78 at a distal end of the elongate shaft, a collar 74 positioned at a proximal end of the blade 78, and an abutment projection 76 extending distally from the collar 74. The abutment projection 76 limits the depth of penetration of the joint probe 78. The blade 78 permits placement of the probe 78 in the articular space and allows the surgeon to manipulate the articular space as needed. The joint probe 70 allows the surgeon to locate the center of the joint, manipulate the articular space, set any desired correction, and assess the joint size and depth. Blade 78 may be a flat blade or a tapered blade. Blade 78 has major surfaces that are substantially planar and are configured to be proximate to articular surfaces of each opposing bone when probe 70 is inserted into a joint. Blade 78 also has minor surfaces that are substantially orthogonal to the major surfaces and are configured to be positioned both medial and lateral to the opposing bones when probe 70 is inserted into a joint. As is shown, infra, with respect to alternative embodiments of probe 70 illustrated in FIGS. 31A to 34, the minor surfaces of the blade 78 may be configured to have a shape corresponding to a taper of the dowel to be inserted into the joint during the arthrodesis procedure.

FIG. 7 illustrates a guide instrument 80 that is placed concentrically over the joint probe 70 to create a fixed position for drilling or reaming the bones bounding the articular space and subsequent dowel placement within the articular space. Guide instrument 80 consists of a handle 82, a sleeve 85 positioned at a distal end of the handle, a sleeve liner or boss 84 concentrically positioned within the sleeve 85, and at least two fixation guides 86 positioned diametrically opposed to each other on an outer circumferential surface of the sleeve 85, each of the at least two fixation guides 86 having a central bore configured to allow passage of a K-wire (Kirschner wire) therethrough. Each of the at least two fixation guides 86 may optionally have at least one distal projection 88 configured to penetrate into bone to fix the position of the guide instrument 80 across the articular space of a joint. The at least one distal projection 88 may, optionally, be formed by a bevel in the distal end of each of the at least two fixation guides. The sleeve liner or boss 84 is preferably made of a material that allows for the guide instrument 80 to be placed over the joint probe 70 such that the collar 74 is concentric within the sleeve liner or boss 84 and permits both rotational, axial, and circumferential movement of the joint probe 70 within the sleeve liner or boss 84 and guide instrument 80. The sleeve liner or boss 84 and/or the sleeve 85 may, optionally, be keyed, such as by a linear projection or recess on an inner diametric surface of the sleeve liner or boss 84 and/or the sleeve 85 that mates with a corresponding recess or projection on the joint probe 70. In this manner, cooperation between the keying surfaces of the sleeve liner or boss 84 or sleeve 85 and the joint probe 70 serves to align and fix orientation of the guide instrument 80 relative to the joint probe 70.

FIG. 8 depicts a K-wire 90 having an elongate body 92, a tapered and pointed distal end 96. A depth limiter 94 is joined to the elongate body 92 and is configured to abut against a proximal end of the at least to fixation guides 96 and drives the at least two fixation guides 86 into an abutting relationship with the bone, thereby embedding the at least one distal projection 88 on each of the at least two fixation guides 86 into the bone when the K-wire is inserted into the bone to its maximum desired depth as indicated by the depth limiter 94. The depth limiter 94 may be a bead, a ferrule, a collar, an enlarged diametric section of the elongate body 92, or other similar device integrated with or attached to the elongate body 92. The K-wire 90 has a beveled terminal distal end that is capable of boring into bone tissue as the K-wire is delivered through each of the at least two fixation guides 86. The elongate body 92 may, optionally, have a threaded section at a distal end of the elongate body 92 and proximate to the tapered and pointed distal end 96. The threaded section facilitates engagement with bone tissue upon rotating the elongate body 92.

FIG. 9 depicts a drill bit 100 in accordance with the present disclosure. Drill bit 100 consists of a shank 102 configured to engage with a collet or other mechanism on a drill to removably secure the drill bit 100 to the drill. A drill shaft 104 extends from the shank 102 and has a drill head 108 at a distal end of the drill shaft 104. Optionally, drill head 108 has both a length, a proximal diameter, and a taper angle corresponding to the height and taper angle of a corresponding dowel for use in a particular joint. Alternatively, drill head 108 may have a tapered and stepped profile with each step having a different diameter. Further, drill head 108 may have no taper angle and a constant diameter along its length. In all embodiments, the drill head 108 has a helical flute that cuts into opposing bone surfaces of a joint and, at a terminal distal end of the drill head 108 the helical flute terminates in a cutting lip and a flank. A depth stop 106 is concentrically positioned about the drill shaft 104 to limit the penetration depth of the drill head 108 during drilling. The drill bit 100 may be cannulated or non-cannulated.

FIG. 10 illustrates a dowel placement tool 110. Dowel placement tool consists of a proximal handle 112, a shaft portion 114, an alignment indicator 116 in a portion of the shaft portion 114, and a dowel engagement 118 at a distal end of the shaft portion 114. The shaft portion 114 is preferably cylindrical and configured to be capable of being passed into and through the sleeve 84 of the guide instrument 80 to allow placement and release of the dowel within the drilled recess created by the drill bit 100 in the articular space of a joint. The alignment indicator 116 may serve as a depth marker indicating the minimum and maximum dowel implant depths within the joint. Alignment indicator 116 may be a circumferential groove in the shaft portion 114, be calendar markings on the shaft portion 114, or be other tactile or visual indicia on the shaft portion 114 that demarcate minimum and maximum depths for the dowel implant.

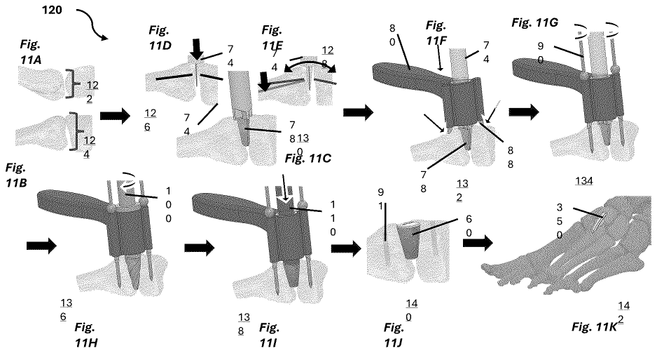

FIGS. 11A to 11K sequentially illustrate steps in an arthrodesis procedure 120 employing a dowel and the arthrodesis instrumentation in accordance with the present disclosure. While the arthrodesis method 120 is depicted with reference a mid-foot joint, such as the TMT joint, it is intended that the arthrodesis method 120 is applicable to other joints, such as in the foot, hand, wrist, or other parts of the anatomy. First, a joint is identified at steps 122, 124, then the wedge-shaped probe 78 is introduced into the articular space and manipulated to expand the articular space at steps 126, 128, 130. Once the articular space of the joint has been expanded, the guide instrument is introduced over the joint expansion tool and seated against opposing bones of the joint in a desired position 132 by embedding the at least one distal projection 88 on the guide instrument into the bones. Once the guide instrument is secured to the opposing bones of the joint, the K-wires 90 are inserted 134 through the at least two fixation guides 86 and into each of the opposing bones of the joint, thereby positionally securing the guide instrument across the articular space of the joint and forming K-wire bores 360 in each of the opposing bones.

Subsequent step 136 entails removing the joint probe 70 with the articular space being held open by the guide instrument 80 and K-wires 90 and then introducing the drill bit 100 through the sleeve liner 84 of the guide instrument 80. The drill bit 100 is then rotated by the drill (not shown) and the drill head 108 simultaneously forms a conical-shaped profile in the opposing bones of the joint abutting the articular space. The conical-shaped profile formed by the drill head 108, corresponds to the size and shape of the dowel to be fixed within the joint 5. The drill bit 100 is then removed from the joint.

Once a dowel opening in the articular space has been created, the dowel delivery device 110 with the dowel removably coupled thereto, is introduced through the sleeve 85 and sleeve liner 84 and the dowel positioned within the dowel opening in the articular space.

The guide instrument and K-wires are then removed at step 140, leaving the dowel in place within the joint. A staple 350 is then placed over the dowel 60 and secured to the opposing bones to retain the dowel in the placed position. The staple 350 may be placed in new blind holes bored into each of the opposing bones or may be placed in the K-wire bores 360 if the staple is of appropriate dimension.

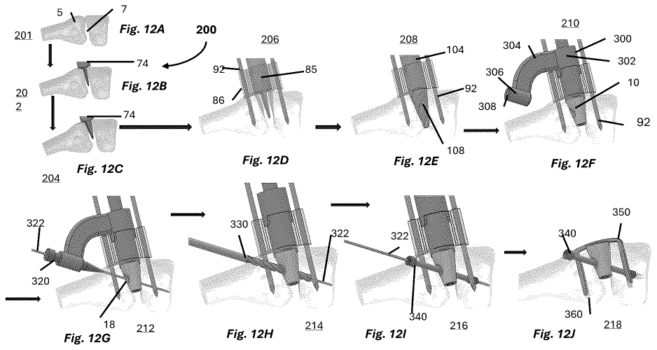

FIGS. 12A to 12J sequentially illustrate steps in an arthrodesis procedure 200 with utilization of each of the arthrodesis instruments to place and fix the arthrodesis dowel in the articular space of a joint in accordance with the present disclosure. Joint identification 201 is first performed to identify the joint 5 in need of joint fusion and the joint 5 is exposed through surgical incision. The joint probe 70 is then introduced into the articular space 7 at step 202 and the wedge-shaped probe 78 of the joint probe 70 is manipulated to manipulate or expand the articular space at step 204. Thereafter, at step 206, the guide instrument 80 is placed over the joint probe 70 with the elongate shaft 72 and collar 74 passing through the sleeve liner 84. The at least two tubular members 86 positioned on opposing bones of the joint 5 and the distal projections 88 on each of the at least two tubular members 86 are secured to the opposing bones of the joint 5 such that the guide instrument 80 spans the articular space 7 of the joint 5. K-wires 92 are then passed through the at least two tubular members 86 and into the opposing bones of the joint 5 to fix the guide instrument 80 across the articular space 7 of joint 5.

Subsequent step 208 entails removing the joint probe 70 with the articular space being held open by the guide instrument 80 and then introducing the drill bit 100 through the sleeve liner 84 of the guide instrument 80. The drill bit 100 is then rotated by the drill (not shown) and the drill head 108 simultaneously forms a conical-shaped profile in the opposing bones of the joint abutting the articular space. The conical-shaped profile formed by the drill head 108, corresponds to the size and shape of the dowel to be fixed within the joint 5. The drill bit 100 is then removed from the joint.

Step 210 entails introducing a dowel corresponding to the size and shape of the conical-shaped profile formed in the joint by passing the dowel removably coupled to the dowel placement tool 100 through the sleeve 85 of the alignment device 80 and seating the dowel into the articular space of the joint. Where the dowel has a traverse angled bore 18, 28, 58, 68, a compression screw 340 is placed across the joint 5 and through the transverse angled bore 18, 28, 58, 68, to fix the dowel within the articular space 7 of the joint. Compression screw 340 may be a constant compression screw that exerts a consistent axial compression of the bones being compressed across the arthrodesis. Alternatively, the compression screw 340 or a dynamic compression screw 340 that dynamically adjusts the axial compression force as forces act on the bones across the joint.

In order to place a compression screw 340, a drill guide 300 employed and passed over the proximal shaft portion 114 of the dowel placement tool 110, positioned concentrically about the distal shaft portion 116 of the dowel placement tool 110 and engaged with the guide instrument 80. Drill guide 300 consists of tubular body 302 that concentrically engages about the distal shaft portion 116 of the dowel placement tool 110, an arm 304 extending radially outward and projecting distally from the tubular body 302, and a tubular guide 306 with a guide bore 308 at the end of the arm 304, wherein the guide bore 308 is in angular alignment with the transverse angled bore 18, 28, 58, 68 of the dowel.

In step 212 an introducer 320 is coupled to the tubular guide 306 by placing it through guide bore 308, and an introducer wire 322 is passed through the introducer 320, through a first bone, through the transverse angled bore 18, 28, 58, 68, and into the opposing second bone. To facilitate passing the introducer wire 322, optionally, a small introducer hole may be drilled through the joint bones in alignment with the transverse angled bore.

FIGS. 12H and 12I illustrate the drill guide 300 with the arm 304 not shown for clarity. Removal of the arm 304 or drill guide 300 prior to deploying cannulated drill bit 330 over the introducer wire 322 is optional. The introducer 320 is then removed, leaving the introducer wire 322 in the joint and the guide instrument 80 secured across the joint in step 214. A cannulated drill bit 330 is then passed over the introducer wire 322 and passed into the first bone, through the transverse angled bore 18, 28, 58, 68, and into the opposing second bone to create a drilled opening to accommodate the compression screw 340.

In step 216, the compression screw 340 is introduced over the introducer wire 322 and engaged within each of the opposing bones of the joint 5 and through the transvers angled bore 18, 28, 58, 68 of the dowel The introducer wire 322, the K-wires 96, the dowel delivery tool 110, and the guide instrument 80 are then removed from the joint and a staple 350 is placed across the proximal surface of the dowel with the staple 350 legs passing into the K-wire openings 360, or other openings, in the opposing bones.

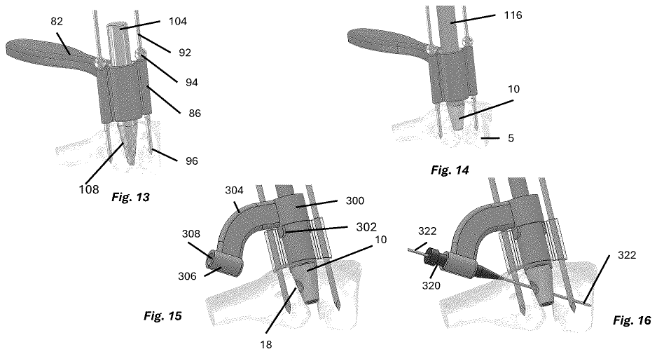

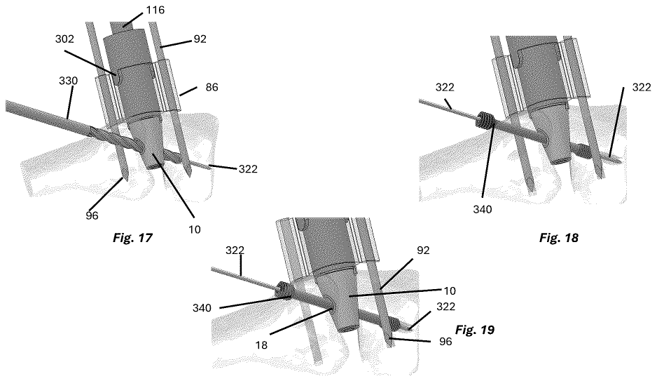

FIGS. 13-21 illustrate the operational engagement between the arthrodesis instrumentation, including the drill bit 100, the guide instrument 80, the K-wires 90, and the drill guide 300, with dowel 10, and joint 5, as they are employed during the arthrodesis procedure 200 depicted in FIGS. 12A-12J, described above.

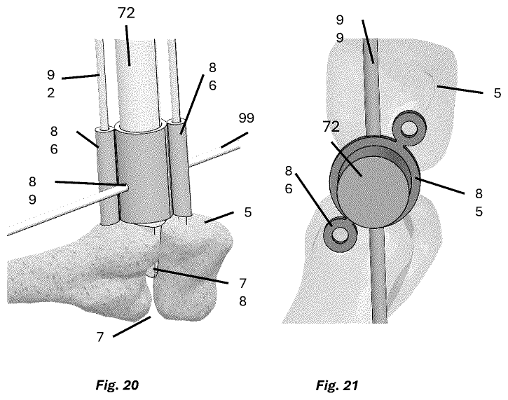

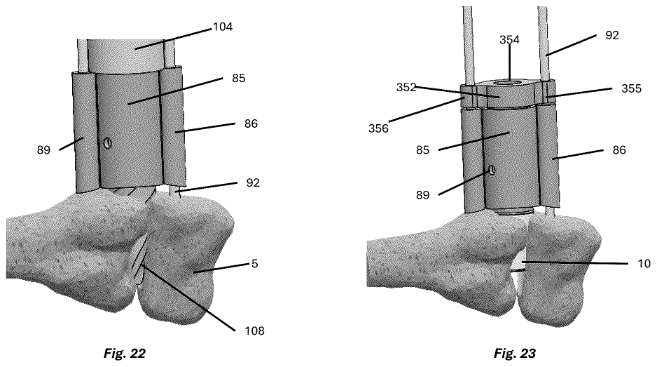

FIGS. 22 and 23 depict alternative views of a variant the guide instrument 80 attached across a joint 5 and spanning the articular space 7 with the joint expansion shaft 72 and wedge-shaped probe 78 being also shown in FIG. 20, the drill bit 104 being also shown in FIG. 22, and the dowel 10 also being shown in FIG. 23. In this variant of the joint alignment device 80, additional alignment openings 89 are provided that pass laterally through the sleeve 85 and sleeve liner 84 and are in diametric alignment with each other. Additional corresponding transverse openings may be provided in the joint expansion tool and the dowel delivery tool to provide consistent alignment and positioning relative to the joint. An alignment probe 99, shown in FIG. 20, is passed through the alignment openings 90 to visualize alignment for drilling and dowel placement.

An optional alignment collar 355 is shown in FIG. 23. Alignment collar 355 consists of a central annular section 352 having a central opening 354 configured to be in axial alignment with the central opening passing through the sleeve 85 of the guide instrument 80. At least two annular projections 356 extend in diametrically opposite directions from the alignment collar 355 and have annular openings configured to be in axial alignment with the bores 87 of the at least two fixation guides 86 passing therethrough that allow the K-wires 92 to pass there through.

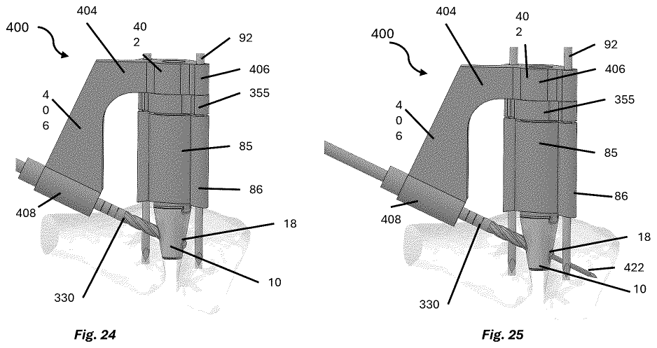

FIGS. 24 to 28 depict steps in placing the compression screw 340 over the introducer guidewire 422 with the guide instrument 80 having the alignment collar 355 coupled thereto.

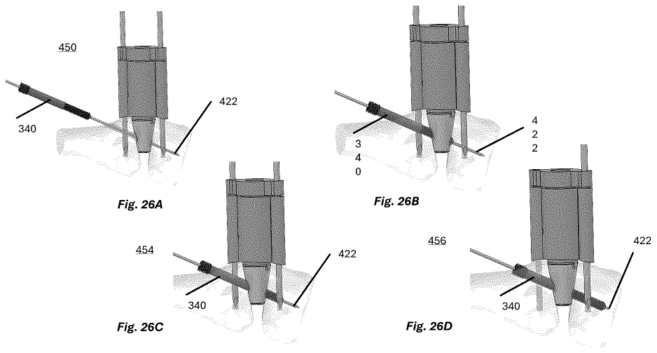

FIGS. 24 and 25 illustrate an alternative embodiment of a transverse drill guide 400. Drill guide 400 consists of tubular body 406 that concentrically engages over the K-wires and abuts the optional alignment collar 355. An arm 404 extends radially outward and has a distally projecting angled portion 406 that terminates in a tubular guide 408 having a guide bore (not shown) that allows a drill 330 to pass there through, the tubular guide 408 has an axial orientation that is positioned transverse and angled in the plantar direction relative to the longitudinal axis of the guide tool 80 and the dowel 10. Arm 404 extends extending radially outward and projecting distally from the tubular body 402, and a tubular guide 406 with a guide bore 408 at the end of the arm 404, wherein the guide bore 408 is in angular alignment with the transverse angled bore 18, 28, 58, 68 of the dowel.

FIGS. 26A to 26D depict sequential steps 450, 452, 454, 456 illustrating placement of a compression screw 340 over the guidewire 422 that is passed through the transverse bore 18 in dowel 10 with the compression screw 340 ultimately seated across the joint 5 into the opposing bones and through the transverse angled bore 18, 28, 58, 68 of dowel 10.

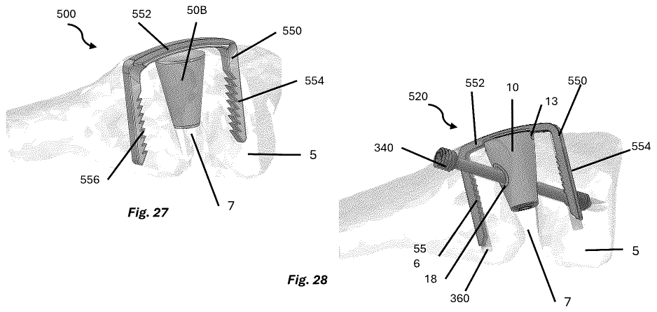

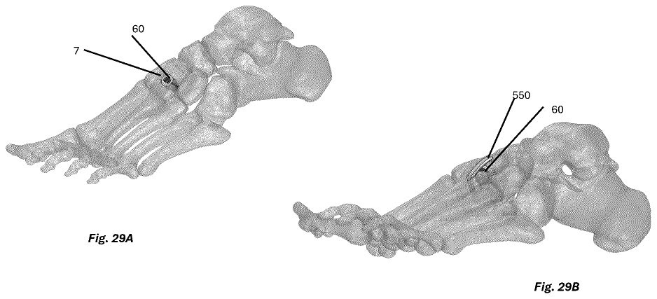

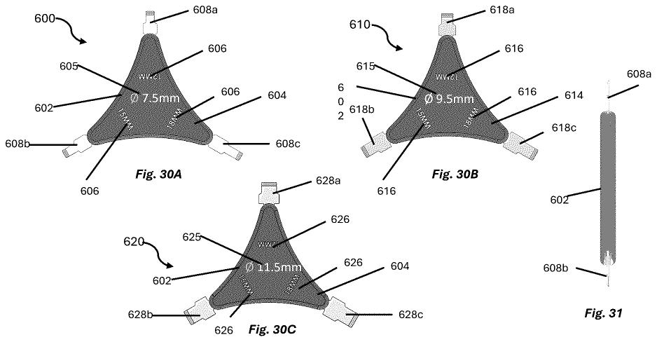

Finally, FIG. 27 and FIG. 28 illustrate adjunctive fixation of the dowel 10 in a joint 5 using a fixation staple 550 configured to seat in each of the opposing bones 5 and secure the dowel 10 within the articular space 7 of the joint 5. FIG. 27 depicts deployment 500 of staple 550 across the joint 5 and spanning dowel 50B. Dowel 50B may or may not have a depression 13, 23, 33 in the proximal or upper surface of the dowel 50B. Staple 550 consists of a staple head 502 and a pair of staple legs 504 positioned at and projecting from opposite ends of the staple head 502. Each staple leg 504 may, optionally, have retention members 56, such as, for examples, ribs, detents, barbs, or other projections from each staple leg 504 that are configured to retain the staple leg 504 in the bone. Retention members 506 may be configured to embed in the bone and prevent pull-out of the staple 500 from the bone into which it is embedded or placed. Once placed, staple head 502 spans the upper or proximal surface of the dowel 50B. Where a depression is present in the upper or proximal surface of the dowel 50B, the staple head 502 engages with and seats at least partially within the depression to prevent retrograde movement of the dowel 50B out of the articular space 7 while the arthrodesis is healing.

A similar staple 520 is illustrated in FIG. 28 which depicts staple 550 having a staple head 502 at least partially engaged with the depression 13 of dowel 10 and the staple legs 504 embedded or placed within K-wire recesses 360 in each of the opposing bones of joint 5. With dowel 10, a compression screw 340 also secures the dowel 10 within the articular space 7 of joint 5 and the staple 520 is positioned to avoid interference with the compression screw 340 both within the bones and outside the bones.

FIGS. 29A-29B illustrate completed placement of dowel 60 in a TMT joint 7 both without adjunctive fixation (FIG. 29A) or with adjunctive fixation (FIG. 29B) with staple 550.

FIGS. 30A to 31 illustrate variants of a joint sizing gauge 600 that differ in dimension, e.g., depth, width, thickness, etc. of the associated gauges 608a, 608b, 608c. Joint sizing gauge 600 may be used as an alternative to or in addition the joint probe 70 to determine the joint size the appropriate sizing of dowel 10. For ease of understanding, reference will be made to a first variant of joint sizing gauge 600 depicted in FIG. 30A. Joint sizing gauge 600 includes a body 602 that serves as a handle and carries at least one, but preferably three dowel gauges 608a, 608b, 608c, at least one dowel gauge indicia 606 corresponding to the diameter and length of each of the dowel gauges 608a, 608b, 608c. Each of the dowel gauges 608a, 608b, 608c, have a substantially planar conformation and a transverse profile corresponding to a transverse profile of a dowel having a diameter and length corresponding to that of the dowel gauge 608a, 608b, 608c, as indicated by the dowel gauge indicia 606. Body 602, while depicted in the accompanying Figures as having a triangular shape with three apices 604, each apex 604 having one of the three dowel gauges 608a, 608b, 608c projecting therefrom, may be configured in a wide variety of manners, including a substantially planar linear shape, a triangular shape, a quadrilateral shape, a pentagonal shape, circular shape, or other shapes etc. Non-planar bodies 602 are also expressly contemplated and intended by the present disclosure. The dowel gauges 608a, 608b, 608c may be affixed portions of the body 602 and project therefrom, as illustrated in the accompanying figures, or may be a handle having plural dowel gauges pivotally attached to the body in a stacked manner that allows the surgeon to select between alternative dowel gauges.

Those skilled in the art will understand that dowel gauges 610 and 620 have like configurations as dowel gauge 600, with dowel gauge 610 having a body 602, at least one of a plurality of dowel gauges 618a, 618b, 618c, and indicia 616, 615 indicating the diameter and length of each dowel gauge 618a, 618b, 618c. Similarly, dowel gauge 620 has a body 602, at least one of a plurality of dowel gauges 628a, 628b, 628c, and indicia 625, 626 indicating the depth and diameter of each dowel gauge 628a, 628b, 628c. Again, each of the dowel gauges 618a, 618b, 618c and 628a, 628b, 628c have a substantially planar conformation and a transverse profile corresponding to a transverse profile of a dowel having a diameter and length corresponding to that of the corresponding dowel gauge. Dowel gauge indicia 602, 605, 612, 615, 622, 625 indicate a corresponding dowel diameter, e.g., 7.5 mm, 9.5 mm, or 11.5 mm and corresponding dowel length, e.g., 12 mm, 15 mm, 18 mm. It will be understood that these dowel diameters and dowel lengths are non-limiting and may be any dowel diameter or length corresponding to the joint anatomy for which the dowel 10 is intended.

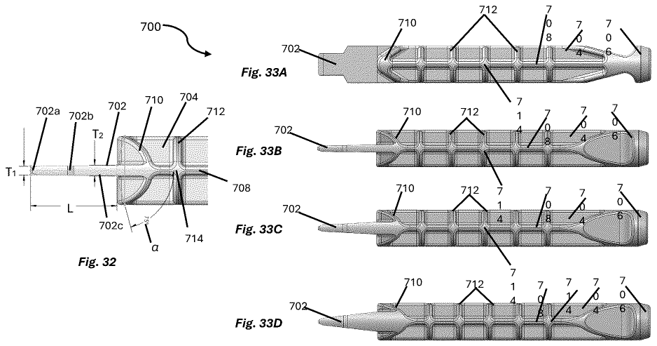

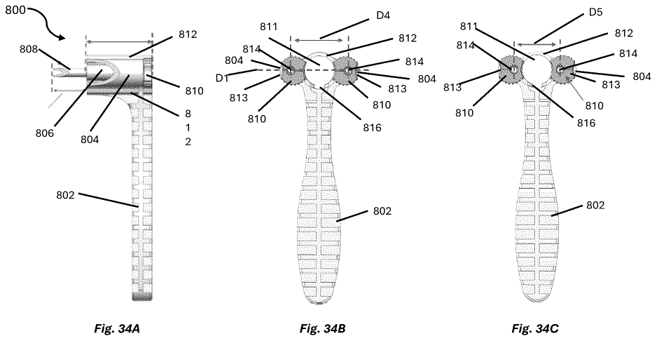

FIGS. 32 and 33A-33D depict dowel probe variants 700 Dowel probe variants 700 differ only in the dimensions their distal end 702 Distal probe end 702 each is configured with a proximal to distal taper, relative to the longitudinal axis of the dowel probe 700, and have substantially planar surfaces with lateral surfaces of the distal probe end 702 being configured to the shape of dowel 10. Each of the distal probe ends 702 have a proximal portion 702c and a distal portion 702a, with a transition portion 702b between the distal portion 702a and the proximal portion 702c. The transition portion 702 replicates the angle of the tapered or stepped transition portion 69 of dowel 10 (as shown in FIGS. 5C and 5D). As illustrated in FIG. 32, a thickness T1 at a distal end of the distal portion 702a of the distal ends 702 is less than a thickness T2 at a proximal end 702c of the proximal portion of the distal probe ends 702. This tapered thickness differential between T1 and T2 facilitates insertion of the dowel probe 700 into the articular space and allows the surgeon to manipulate the joint as desired prior to drilling and dowel placement.

Each of the dowel probe variants 700 have a shaft 704, respectively, to which the distal probe end 702 is attached to or projects axially therefrom. A handle 706 is provided at a proximal end of the dowel probe 700. A plurality of circumferential ribs 712 and longitudinal ribs 708 are provided on the shaft 704 to aid in reinforcing the shaft 704 and allow for grip surfaces for the surgeon. The plurality of circumferential ribs 712 and the plurality of longitudinal ribs 708 intersect at intersection points 714. An arcuate or sinusoidal rib 710 circumscribes a circumference of a distal end of shaft 704 and joins with a proximal end of the distal probe end 702. An angle α is created between a distal most intersection point 714 and a distal most intersection between a longitudinal rib and the arcuate or sinusoidal rib 710. Arcuate or sinusoidal rib 710 further serves as a visual clocking indicator of the positioning of the distal probe end 702.

FIGS. 34A-34C depict an alternative embodiment of the guide instrument 800 in which there is a handle 802, a sleeve 812 having a central opening 811 positioned at a distal end of the handle 802, and at least two fixation guides 804 positioned diametrically opposed to each other on an outer circumferential surface of the sleeve 812. Each of the at least two fixation guides 804 having a central bore configured to allow passage of a K-wire (Kirschner wire) therethrough. An adjustable dial 810 may, optionally, be coupled to a proximal end of each of the at least two fixation guides 804. Each adjustable dial 810 has an offset opening 814 and a pair of diametrically opposed detents or recesses 813 in an outer peripheral surface of the adjustable dial 810. The detents or recesses 813 engage with a portion of the sleeve 812 to fix a position of the adjustable dial 810 and the offset openings 814. Adjustable dial 810 is rotatable such that the offset openings 814 rotate and laterally adjust both inward and outward such that the distance between the offset openings 814 adjusts between a first distance D4 and a second distance D5, that is different than the first distance D4. In this manner, the distance between the offset openings may be adjusted to accommodate different anatomical spacing across a joint.

Each of the at least two fixation guides 804 may optionally have at least one distal projection 808 configured to penetrate into bone to fix the position of the guide instrument 800 across the articular space of a joint. The at least one distal projection 808 may, optionally, be formed by a bevel in the distal end of each of the at least two fixation guides 804. The sleeve 812 may, optionally, be keyed, such as by a linear projection or recess 816 on an inner diametric surface of the sleeve 812 that mates with a corresponding recess or projection on another instrument, such as the joint probe 700, the drill 850, or the dowel driver 900, that passes through the central opening 811 in sleeve 812. In this manner, cooperation between the keying surfaces 816 of sleeve 812 serves to align and fix orientation of the guide instrument 800 relative to another arthrodesis instrument.

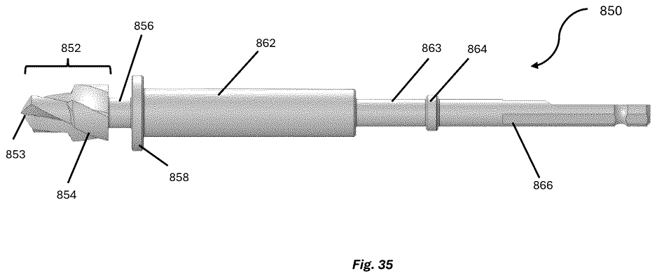

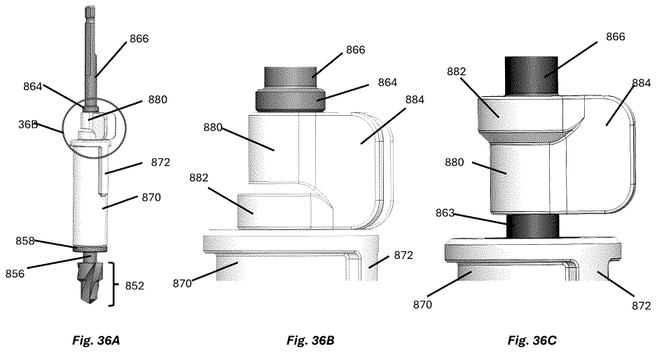

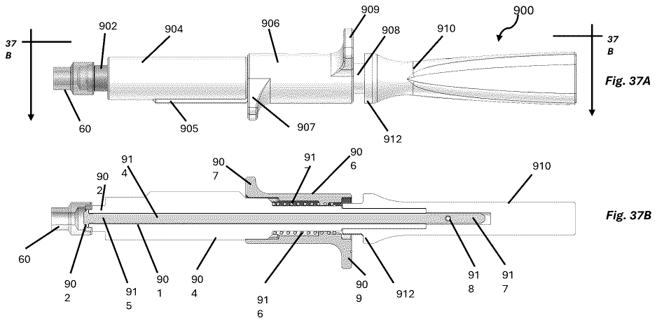

FIG. 35 depicts a drill bit 850 configured to drill or burr bone and create a dowel-shaped opening in the joint for insertion of the dowel 10. Drill bit 850 consists generally of a drill or burr head 852 that is configured to have a first drill portion 353 and a second drill portion 354 wherein the drill or burr head 852 has a conformation corresponding to the conformation and dimension of a dowel 10 in which the first drill portion 353 has a smaller diameter than the second drill portion 354. The first drill portion 353 and the second drill portion 354 have flutes and land areas that may have the same periodicity and configuration of different periodicity and configuration. Immediately proximal to the drill or burr head is a recess 856 that allows bone fragments to be released from the drill bit 850 during a drilling operation. A depth stop 858 is provided proximal to the recess 856 and consists of a circumferential abutment that serves both to delimit the depth of drilling as well as abut a distal end of a drill housing 870, shown in FIG. 36A. Drill bit 850 has a multi-part shank, with a distal shank portion 862 having an enlarged diametric profile that concentrically mates within the drill housing 870, an intermediate shank portion 833, a proximal stop 864 concentrically positioned about the multi-part shank, and a distal shank portion 866 configured to engage with a drill or burr rotary driver (not shown).

As shown in FIGS. 36A-36C, the drill bit 850 couples with the drill housing 870, with the drill housing abutting the depth stop 858 and leaving the drill or burr head 852 and the recess 856 exposed at a distal end of the drill housing 870. Drill housing 870 optionally has a clocking or alignment protrusion or recess 872 at a proximal end of the drill housing 870. Alignment protrusion or recess 872 may extend along a longitudinal aspect of the drill housing 870 and be configured to mate with the alignment protrusion or recess 816 in the guide instrument 800. A clip 880 is provided and removably engages the intermediate shank portion 862. Clip 880 operates as a spacer between the proximal stop 864 and a proximal end of the drill housing 870 to delimit the depth that the drill or burr head 852 can penetrate into bone. Clip 880 has ears 884 that allow insertion or removal of clip 880 from the intermediate shank portion 862. Clip 880 is reversible to allow for additional drilling depth. In the reversed position, a distal end 882 of clip 880 is positioned proximally with the proximal stop 864 nested within the distal end 882 of clip 880 such that a section of the intermediate shank portion 862 is open and a space is created between the clip 880 and the proximal end of the drill housing 870. In this reversed position, the drill bit 850 may be longitudinally translated the distance of the created space to provide a greater depth of drilling into the bone.

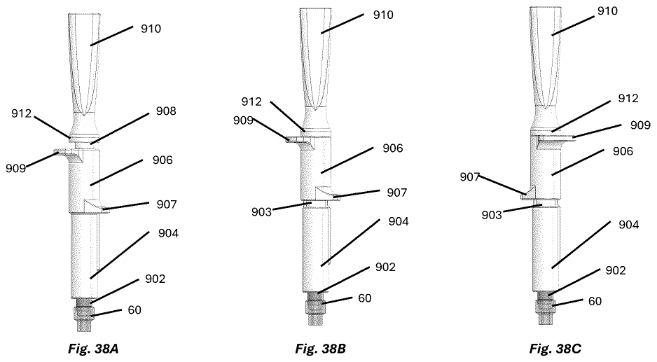

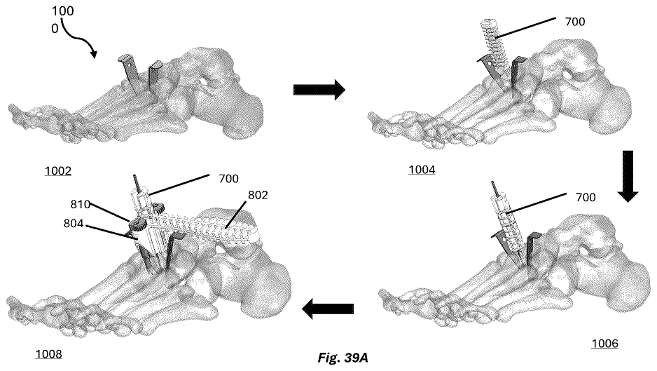

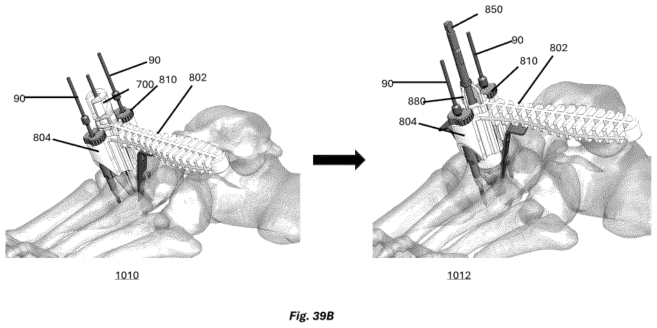

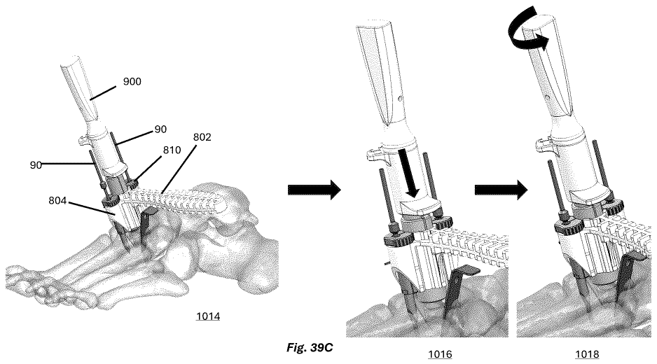

A dowel driver 900 is illustrated in FIGS. 37A to 38C. The drill driver 900 is an instrument that removably couples to a dowel 60 to both deliver, position, and release the dowel 60 in a drilled and prepared joint. The dowel driver 900 consists generally of a mandrel 914 that extends along a central longitudinal axis of the dowel driver 900 and terminates in a dowel coupling 902 at a distal end of the mandrel 914. A handle 910 is coupled to a proximal end 917 of the mandrel 914. The coupling between the handle 910 and the mandrel 914 may be made by any of a large number of suitable methods, such as an aperture 918 passing through the mandrel that aligns with an opening (not shown) in handle 910 and a coupling pin (not shown) that couples the aperture 918 with the opening in handle 910 to couple the handle 910 to the mandrel 914. Coupling 902 is configured to allow for mating with and releasing the dowel 60 upon at least a quarter-turn of coupling 902 and mandrel 914. Coupling 902 may be configured in a generally T-shape with diametrically projecting legs or ears that engage with coupling aperture 67 in dowel 10.

Dowel driver 900 further includes a mandrel housing 904 having a central bore 901 through which the mandrel 914 passes. An actuating member 906 having a distal handle 907 and a proximal handle 909 is concentrically engaged about a proximal aspect of the mandrel housing 904 and a distal aspect of the handle 910. A traveler 916, such as a spring or a helical recess and mating traveler pin, is provided between the mandrel housing 904 and the actuating member 906. The traveler 916 allows for axial compression of the handle 910 relative to the mandrel housing 904 and rotation of the handle 910 to rotate the coupling 902 to engage or disengage with the dowel 60. Alternatively, as depicted in FIGS. 38A-38C, the mandrel housing 904 may be axially translated proximally to rest against a distal abutment 912 of the handle 910, then the mandrel housing rotated circumferentially to either engage or disengage the coupling 902 with respect to the dowel 60.

Finally, Figures, 39A-39C depict an arthrodesis method 1000 for implanting dowel 10 using the above-described arthrodesis instrumentation, including dowel probe 700, guide instrument 800, fixation wires 90, and dowel driver 800. Arthrodesis method 1000 starts with access an orthopedic joint, such as a mid-foot joint. Illustrated for exemplary purposes, is the TMT joint of the mid-foot anatomic architecture. Once the joint is accessed at lest one joint finder device, as described in U.S. Patent Application Publication No. 2024/0260852, published Aug. 8, 2024, which is hereby incorporated by reference in its entirety, is employed at step 1002 to locate and manipulate the joint for introduction of the dowel probe 700 into the joint. Thereafter an alignment wire is introduced at step 1006 and then the guide instrument 800 is placed over the dowel probe 700 and the alignment wire and positioned across the joint. The adjustable dials 810 are adjusted to align the bores of the fixation guides with the opposing bones in a desired position, then fixation wires 90 are then inserted through the fixation guides 804 and into the opposing bones of the joint at step 1010. Once the fixation wires 90 are placed, the dowel probe 700 is removed, and the drill bit 850 and drill housing 870 are passed through the central opening 811 of sleeve 812 of the guide instrument 800, and the drill bit 850 actuated to drill a dowel-shaped bore in the joint at step 1012. Once the dowel-shaped bore is formed, the drill housing 870 and drill bit 850 are removed from the guide instrument 800 and the dowel driver 900 is introduced through the central opening 811 or sleeve 812 of the guide instrument 800 and the dowel 60 positioned within the dowel-shaped bore in the joint and the dowel 60 is delivered by releasing it from the dowel driver 900 at steps 1014, 1016, and 1018.

Those skilled in the art will understand and appreciate that modifications in dimensions, materials, configurations, and methods of creating the arthrodesis using the above-described devices and systems may be made without departing from the scope of the disclosure, which is intended to be limited only by the claims appended hereto.

1. An arthrodesis dowel, comprising a frustoconical dowel having a porous body made of a biocompatible material, a non-porous proximal surface, and an opening in the non-porous proximal surface configured to removably couple to a dowel delivery device, wherein the frustoconical dowel is configured to be placed within a bore between adjacent resected bones.

2. The arthrodesis dowel of claim 1, wherein the frustoconical dowel further comprises a distally tapering wall surface extending between the non-porous proximal surface and a distal surface of the frustoconical dowel.

3. The arthrodesis dowel of claim 2, wherein the non-porous proximal surface further comprises a coupling opening.

4. The arthrodesis dowel of claim 1, wherein the side walls are stepped side walls.

5. The arthrodesis dowel of claim 4, where in the biocompatible material is selected from the group of titanium, stainless steel, cobalt chrome, nickel-titanium alloys, tantalum, polyetheretherketone (PEEK), magnesium, silicon nitride, alumina, zirconia, poly(methyl methacrylate) (PMMA), polylactic acid (PLA), carbon fiber-polyetherketone, or carbon fiber-ultrahigh molecular massed polyethylene.

6. The arthrodesis dowel of claim 4, wherein the frustoconical dowel further comprises a bore passing longitudinally between the non-porous proximal surface and the distal surface.

7. The arthrodesis dowel of claim 4, wherein the distally tapering wall surface is porous.

8. The arthrodesis dowel of claim 4, wherein the stepped side walls further comprise a proximal section having a first diameter and a distal section having a second diameter, wherein the first diameter is greater than the second diameter.

9. The arthrodesis dowel of claim 8, wherein the stepped side walls are porous throughout a thickness of the side walls.

10. A joint arthrodesis dowel, comprising frustoconical dowel a substantially planar proximal surface, a substantially planar distal surface, and side walls extending between the substantially planar proximal surface and the substantially planar distal surface, wherein the substantially planar proximal surface has a diameter larger than a diameter of the substantially planar distal surface and the side walls are made of a porous material having a porosity configured for osteointegration and the substantially planar proximal surface is made of a non-porous material.

11. The joint arthrodesis dowel of claim 10, further comprising a central bore passing between the substantially planar proximal surface and the substantially planar distal surface.

12. The joint arthrodesis dowel of claim 11, wherein the substantially planar proximal surface further comprises a coupling opening passing there through, the coupling opening communicating with the central bore.

13. The joint arthrodesis dowel of claim 10, wherein the side walls are stepped side walls.

14. The joint arthrodesis dowel of claim 13, wherein the stepped side walls are each co-axial with a longitudinal axis of the joint arthrodesis dowel.

15. The joint arthrodesis dowel of claim 14, wherein the stepped side walls each have an outward distal taper or an inward distal taper relative to a central longitudinal axis of the arthrodesis dowel.

16. The joint arthrodesis dowel of claim 13, wherein the stepped side walls are porous throughout a thickness of the stepped side walls.

17. The joint arthrodesis dowel of claim 10, where in the biocompatible material is selected from the group of titanium, stainless steel, cobalt chrome, nickel-titanium alloys, tantalum, polyetheretherketone (PEEK), magnesium, silicon nitride, alumina, zirconia, poly(methyl methacrylate) (PMMA), polylactic acid (PLA), carbon fiber-polyetherketone, or carbon fiber-ultrahigh molecular massed polyethylene.

18. The joint arthrodesis dowel of claim 10, wherein the side walls have a roughened surface topography.

19. The joint arthrodesis dowel of claim 10, wherein the side walls are formed of more than one taper angle.

20. The joint arthrodesis dowel of claim 10, further comprising a retention member configured to span the proximal surface of the frustoconical dowel and embed into opposing bones of a joint.
